(19) United States
(12) Patent Application Publication
(10) Pub. No.: US 2014/0148708 A1
(43) Pub. Date: May 29, 2014

(54) SYSTEM AND METHOD FOR WOUND MANAGEMENT

(71) Applicant: Cardiocom, LLC, Chanhassen, MN (US)

(72) Inventors: Daniel L. Cosentino, Excelsior, MN (US); Brian A. Golden, Eden Prairie, MN (US)

(73) Assignee: Cardiocom, LLC, Chanhassen, MN (US)

(21) Appl. No.: 14/091,103

(22) Filed: Nov. 26, 2013

Related U.S. Application Data
(60) Provisional application No. 61/729,778, filed on Nov. 26, 2012.

(51) Int. Cl. A61B 5/00

(52) U.S. Cl. CPC A61B 5/0077; A61B 5/445
USPC 600/476; 600/587

(57) ABSTRACT

A wound management reference object comprising a sheet of material of a known size including at least one colored segment, wherein the wound management reference object is configured to be positioned in close proximity to a wound, wherein an image can be captured of the wound management reference object and the wound, and wherein the image is used to evaluate the wound.

This application claims the benefit of U.S. Provisional Application No. 61/729,778 filed Nov. 26, 2012 which application is incorporated herein by reference.

### INTRODUCTION

Professional healthcare providers are not always available to accommodate a patient for a medical examination immediately after a wound infliction to assess the severity of the wound. In fact, it often takes several days for a patient to schedule a medical appointment that is convenient for both the healthcare provider and the patient. In some instances, images of wounds may be transmitted to healthcare providers for assessment at a remote site; however, image quality issues may often skew healthcare providers' assessments of wounds.

### SYSTEM AND METHOD FOR WOUND MANAGEMENT

A system and method for wound management is described that allows a patient to remotely capture an image of a wound, injury, lesion, cut, gash, sore, abrasion, laceration, or the like, and transmit the image to a healthcare professional for assessment. To assist in the assessment, a reference object for color and size is including in the image to provide a reference for the healthcare professional. With the assistance of the reference object, a healthcare professional can more accurately analyze the image and diagnose the wound and potentially collect and store images of wounds for later use.

In an embodiment, a wound management reference object is provided, wherein the wound management reference object comprises a sheet of material of a known size including at least one colored segment; wherein the wound management reference object is configured to be positioned in close proximity to a wound; wherein an image can be captured of the wound management reference object and the wound; and wherein the image is used to evaluate the wound.

In an alternative embodiment, a wound management system is provided, wherein the system comprises a first reference object, the first reference object comprising a sheet of material of a known size including at least one colored segment; a camera for capturing an image of the first reference object in close proximity to a wound on the individual; and a processor configured to correct the image for at least one of color and size based on the first reference object.

In a third embodiment, a method of wound management is provided, the method comprising providing at least one reference object to a user; directing the user to place the at least one reference object in close proximity to a wound; directing the user to capture an image of the at least one reference object and the wound; and receiving the image at a monitor.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The following drawing figures, which form a part of this application, are illustrative of described technology and are not meant to limit the scope of the invention as claimed in any manner, which scope shall be based on the claims appended hereto.

FIG. 1 depicts one embodiment of a wound management system.

FIGS. 2A and 2B depict alternative placements of a wound management patch.

FIGS. 3A through 3F disclose several embodiments of many possible configurations of a wound management patch.

FIG. 4 illustrates an embodiment of a method for wound management.

### DETAILED DESCRIPTION

The embodiments described herein are implemented as an electronic system and method capable of monitoring a patient and transmitting data to a healthcare professional. Based on the data, the healthcare professional diagnoses, recommends or provides medical treatment, and/or communicates with the patient as necessary. More specifically, a system and method for wound management is described that allows a patient to remotely capture an image of a wound, injury, lesion, cut, gash, sore, abrasion, laceration, or the like, and transmit the image to a healthcare professional for assessment. To assist in the assessment, a reference object for color and size is included in the image to provide a reference guide for the healthcare professional. With the assistance of the reference object, a healthcare professional can more accurately analyze the image and diagnose the wound and potentially collect and store images of wounds for later use.

To properly assess a wound, it is important to be able to accurately perceive the size and color of the wound. When wounds are assessed by analyzing an image of the wound, determining the actual size and color of the wound can be challenging due in part to the display settings of the camera taking the image and/or the display settings of the device displaying the image for the healthcare professional. Body parts and features (hair, dermal ridges, appendages, etc.) can be used for size references if necessary, however these objects do not rectify the problem associated with color discrepancies as described herein, nor do they necessarily provide the healthcare professional with accurate size estimates of the wound. Accordingly, for a more accurate determination of size and color, a reference object having known dimensions and standard colors is more reliable in assisting the healthcare professional with assessing the wound. A reference object of standard size and color that allows for correction of color and determination of size of the wound can be particularly useful for wounds that are analyzed remotely from images. The reference object, as disclosed herein, is positioned next to the wound so that an image of the wound includes both the wound and the reference object. When the image is subsequently transmitted to a recipient with knowledge of the actual dimensions and color of the reference object, the recipient can use this knowledge to properly assess the color, shape, and size of the wound. In so doing, the healthcare provider can compare the color of the wound to a standard reference color on the reference object to determine the severity of the wound and/or the type of wound. Alternatively, the recipient can to correct the image for color, shape, and size. For example, the recipient may, either manually or by using computer software particularly developed for the purpose, adjust the display settings and/or the image such that it is corrected to display the true colors and size of the wound to the recipient's eye.

The system and method for wound management may be particularly useful in assessing and managing wounds of chronic patients, ambulatory patients, home care patients, patients in a care facility, accident victims, or the like. For example, patients with chronic conditions (e.g., diabetic wounds) may benefit from more frequent monitoring of their wounds. Because wounds in such patients are often recurrent, a remote wound assessment benefits such patients by having to less frequently visit a healthcare facility for each occurrence of a wound. With proper support, many such patients can be cared for at home either by themselves or by a caretaker, or at a care facility that does not have access to an on-site physician. Additionally, parents often have to manage injuries of their children and make decisions regarding whether to care for the injury at home or to have the child seen by a physician. Accordingly, a system and method that assists in making decisions about the care of a patient instead of visiting a healthcare facility is useful.

The system and method described herein may be used directly by a patient or accident victim, or indirectly by a care provider, family member of the patient, or another helper. For example, the system and method may be used by a care provider at a facility (e.g., nursing home or assisted living facility), by a home care provider (e.g., a family member), or by a first responder at the scene of an accident. For purposes of the below description, a “user” is a person utilizing the system at the patient location. In some embodiments, the user may receive instructions for wound management and/or patient care or first aid information. Further, the user may inquire if a visit to a hospital is necessary, or may provide advance notice to a care facility (e.g., hospital) of an incoming patient with a wound or injury.

In some embodiments, the reference object is a patch (e.g., a wound management patch) that can be adhered to skin in close proximity to (e.g., next to) a wound. The patch may be constructed of flexible or rigid material (e.g., plastic) and may include a temporary adhesive on the back side. The patch may be disposable or reusable, and may be packaged individually and/or wrapped similarly to an adhesive bandage.

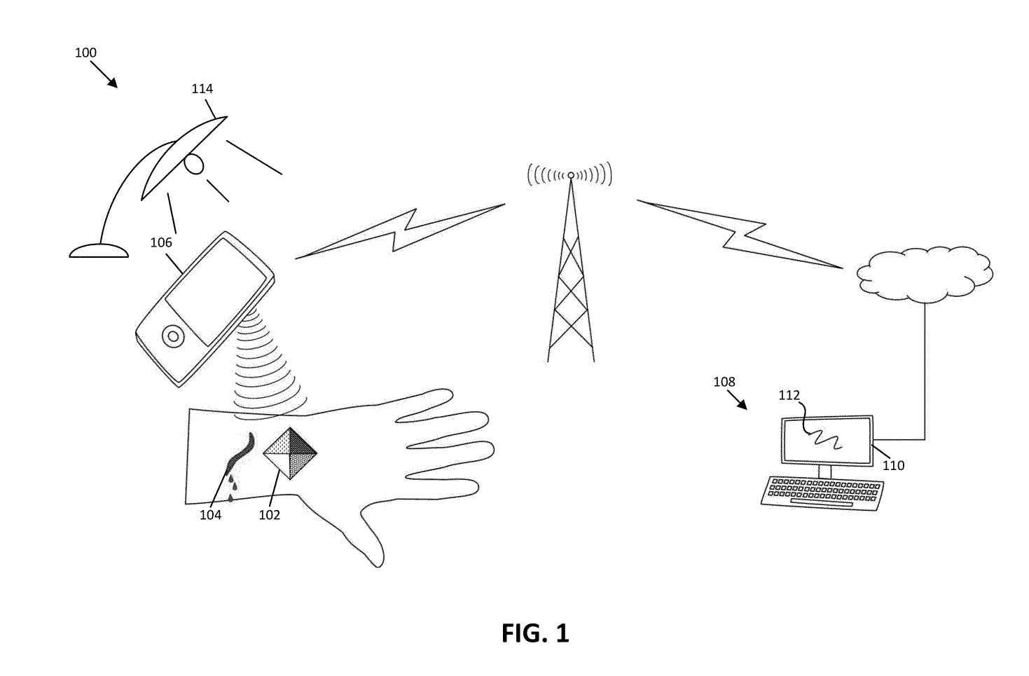

FIG. 1 depicts one embodiment of a wound management system 100. A wound management patch 102 may be adhered to skin, clothing or otherwise in close proximity to a wound 104 that needs assessment and/or treatment. An image of the wound and the patch 102 can be captured using any device 106 capable of taking pictures, such as a camera (digital camera, analog camera or video camera), a cell phone, a smartphone (e.g., an IPHONE available from Apple Inc. in Cupertino, Calif.; a BLACKBERRY available from Research In Motion in Waterloo, Ontario, Canada; or GALAXY available from Samsung Electronics Co. Ltd. in Ridgefield Park, N.J.), a tablet personal computer (PC) (e.g., an IPAD available from Apple Inc. in Cupertino, Calif.; or GALAXY available from Samsung Electronics Co. Ltd. in Ridgefield Park, N.J.), a PC, or a monitoring device in a telehealth system. According to the embodiment, the image can be transmitted to a remote location 108 such as a remote healthcare provider workstation and/or a central processing unit for further processing. The image may then be processed, stored, transmitted to another location, and/or analyzed. Feedback regarding wound management, patient/self-care, or first aid may thereafter be transmitted back to the patient or caretaker using the device 106 or another device capable of receiving such patient wound information. Alternatively, the image may be transmitted to a central processing unit (not shown) where the image is automatically corrected based on the reference object. The corrected image may then either be stored in the central processing unit for later retrieval or automatically sent to a remote healthcare provider. In yet alternative embodiments, the image may be first corrected by software stored on the patient device.

In yet another alternative embodiment, the display setting of a display 110 at the remote location 108 may be manipulated in order to cause the display 110 to render the image so that the patch 102 of the displayed image 112 closely matches the actual patch's 102 colors and size. For example, in an embodiment, the user at the remote location 108 may be provided with a copy of the patch 102 which allows the user to manually adjust the display settings in order to make the patch 102 in the displayed image 112 match the actual patch 102, positioned proximate to the wound 104, as close as possible. Alternatively, the system at the remote location 108 may be provided with feedback regarding how the image is displayed and, based on the feedback, may automatically adjust the display settings or the image in order to match the actual patch's 102 colors as closely as possible.

Various technologies may be utilized by the system and method to transmit, analyze and/or store the image and related data. Some or all of the processing of the image may be done locally at the patient location by using software either on a device 106 such as a handheld device, a computer or telehealth device at the central processing unit, or a remote healthcare provider workstation. For example, upon capturing the image of the wound and the reference object 102, a mobile or web-based application may be used to recognize the reference object 102 (e.g., wound management patch), correct the image for size and color, and analyze the size and color of the wound 104. This information may then be used by the healthcare provider, first responder, caretaker, or the patient, or it may alternatively be transmitted using the handheld device 106 or may be uploaded directly to a database or website (e.g., a remote telehealth system or service). A self-help application may be provided to assist the user with using the information in the care of the patient. The image (and optionally additional data) may be transmitted using any one of available technologies, such as, but not limited to, text messaging (SMS or MMS), e-mail, instant messaging, and/or uploading to a website that is accessible by the healthcare provider. Additional data transmitted (either along with the image or separately) may include, for example, patient identifying information (e.g., name, address, phone number, etc.), patient medical information (e.g., age, weight, pre-existing conditions, allergies, etc.), patient status (e.g., level of consciousness, level of pain, etc.), insurance information, healthcare provider information (e.g., family physician), and/or a time and date stamp when the image was taken.

The image and related data may be stored, for example, in a patient care file or in a database, such as a wound library. According to an embodiment, the image is corrected for size and color prior to storing in a database, and/or may be corrected at the time it is retrieved from the database. A wound library may include, for example, patient information (e.g., personal information and health history), images of previous wounds, prior wound diagnoses, and/or prior wound treatment and healing information. The data in the wound library may also be shared between healthcare professionals. The image and related data may be used subsequently for deciding care decisions, predicting wound healing, or the like. In addition, the data may be used for insurance purposes. Wound images and non-identifiable patient data (e.g., data about the wound, its diagnosis, care and healing) can be stored and used subsequently for educational or research purposes or to develop future improvements to the system or devices (e.g., improvements to automatic image recognition, analysis and diagnosis of wounds). Information stored in a patient care file or a wound library may be used in conjunction with a clinical pathways management system to provide guidance for wound management, patient care, first aid response, or to predict the outcome of the healing of a wound.

A clinical pathways management system, which follows pre-installed parameters may automatically analyze the size/color of the wound or compare the development of the wound (e.g., size or color) over time and provide care instructions, recommendations or alerts depending on the results such comparisons. The clinical pathways management system may be implemented in the patient monitoring system located at the patient site or at the remote site 108. The parameters can be determined by a healthcare professional in charge of caring for the patient and are installed in the system or application such as a self-help application used by the patient. The clinical pathways management system, which follows the predetermined patient parameters, may provide different instructions for different types of wounds. For example, the clinical pathways management system may provide different instructions for a wound that is less than ½ inches in diameter compared to a wound that is ½ to 1 inches or more than 1 inch in diameter. Alternatively, different instructions may be given for a wound with a red color than for a wound with a green or yellow color. Individualized instructions may also be given if the wound size changes, e.g., enlarges or fails to reduce by a certain percentage within a predetermined time period, if the color of the wound changes, and/or if the wound becomes infected.

The system and method disclosed herein may additionally include a wound tracking system. According to some embodiments, information about a patient's wound is stored for later use, for example, to track the healing and/or the development of a chronic condition, such as bed sores, diabetic wounds, or the like. Multiple images of a wound may be captured and stored over time, providing a sequence of images that allows for tracking the development (e.g., healing, changing, or worsening) of the wound. For example changes in size and color of the wound can be tracked. Images of a wound can be added to a wound tracking system either manually or automatically. The progression of the wound/timeline of images can be viewed and analyzed by a medical professional or can be analyzed using software. The images can be viewed separately, on a timeline, or can be compiled into an animated video.

In alternative embodiments, the system includes the use of a standardized light applicator 114 that delivers light at specific wavelengths to help produce accurate colors of the reference object 102 and the wound 104. The system and method may also include an image sensing system (not shown) that generates a normalized (i.e., corrected for color and size) image of the reference object and wound. For example, a patient may place a wound management patch 102 in close proximity to a wound 104 and set up a light applicator 114 to direct light on the area of the wound 104 and reference object 102 before capturing an image utilizing a camera or other image capturing device 106. The camera or other image capturing device 106 may include an image sensing system, or the image sensing system may be part of a separate device (such as a computer or the like at the patient location or at the remote location 108). The image sensing system may automatically correct the image for color and size by comparing the size and color of the wound management patch 102 to pre-loaded data. The corrected image may be used locally to make decisions about care and/or may be transmitted to a remote location 108 for further processing.

According to other alternative embodiments, if the image is captured and transmitted using a handheld image capturing device 106 that is capable of detecting its position using a Global Positioning System (GPS), the global position of the image may also be transmitted with the image. This may be particularly useful in accident situations when persons involved in the accident are not able to articulate their exact location because of unfamiliarity with the area or a lack of street signs or landmarks. For example, an accident victim, a helper, or a responder may capture an image of a wound 104 along with a wound management patch 102 using for example, a smartphone or any other portable computer, and transmit the image and the corresponding GPS coordinates to an emergency response center (e.g., emergency dispatch) located nearby. This may provide emergency medical services with the exact location of the accident, a notice of what types of care might be needed at the scene and/or the types of units that should be dispatched (e.g., whether a medical helicopter is needed), and allows emergency personnel to provide first aid instructions to persons on the scene. The image and GPS coordinates can be transmitted, for example, by using a mobile application that detects the position of the mobile device 106 using the device's GPS capability. In other embodiments, if GPS capability is not available, the image coordinates may be determined using positional data acquired by cell phone tower triangulation and the image may be transmitted to the emergency response center over a cellular network.

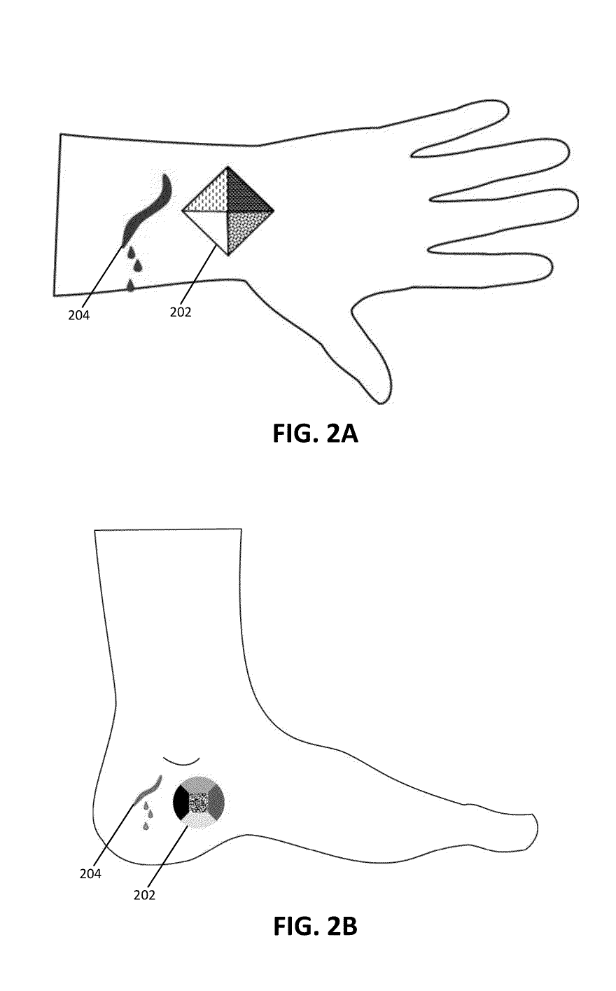

FIGS. 2A and 2B depict alternative placements of a wound management patch 202. As described herein, a wound management patch 202 is be used to assess wounds 204 in various areas of the body, e.g., the extremities, the torso, the neck, or the head. In fact, the patch 202 may be used at any location on the body, or held near the body, as long as the patch is in close proximity of the wound as presented in the image. The patch may be adhered to the skin of the patient, the clothing of the patient, or bandaging covering the area of the body next to the wound. In alternative embodiments, the patch does not include an adhesive, and instead is simply positioned near the wound.

In some examples, the patch 202 is placed in close proximity to the wound 204 such that an image of both the wound 202 and the patch 204 can be captured without substantial size torsion caused by perspective. As used throughout the specification, “close proximity” is used to mean a distance of less than about two to six inches from the edge of the wound. For example, in FIG. 2A, a wound management patch 202 is positioned on the skin of a patient's hand 206 next to a wound 204 so that an image may be captured that includes both the wound 204 and the patch 202. In FIG. 2B, a wound management patch 202 is positioned on the skin of a patient's foot 208. It is understood, however, that in some instances, it may be necessary for the wound management patch 202 to be positioned nearer or farther from the wound.

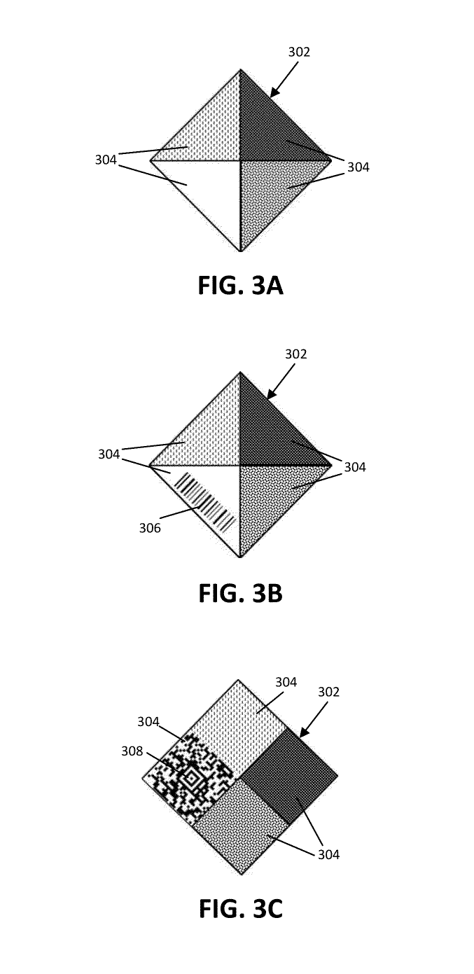

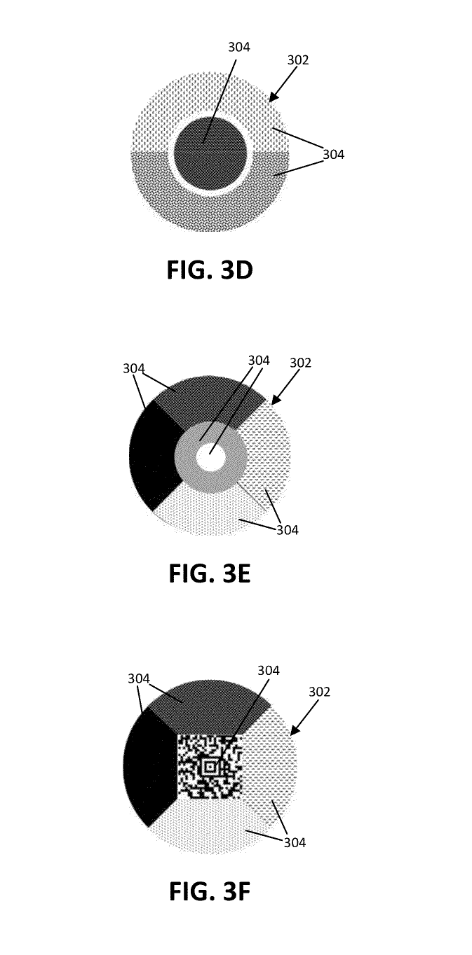

FIGS. 3A through 3F disclose several embodiments of many possible configurations of a wound management patch 302. For example, a square/rectangle/diamond shape can be used, as in FIGS. 3A through 3C, or an oval/round shape can alternatively be used, as in FIGS. 3D through 3F. Other shapes are also possible, and the possible shapes of the wound management patch 302 are not limited to the particular shapes of the patches 302 shown in the figures.

As shown in FIGS. 3A through 3C, the patch 302 includes multiple color segments 304, but may alternatively include only one color segment 304. As described herein, the color segments 304 are used to assist a healthcare provider in properly assessing the wound. The colors may be specified as standard colors so that different devices can normalize the display. For example, each color may be specified in the CMYK standard, the Pantone standard, the RGB triplet format, hexadecimal triplet format or any other known standard.

As shown in FIGS. 3A-3F, the patch 302 is divided into multiple color segments 304, one of which may be white, as shown in FIGS. 3A, 3B, and 3E. For example, the patch 302 may include one or more of: a red segment, a blue segment, a green segment and a white segment. The patch 302 may also include one or more of: a black segment, a magenta segment, a cyan segment, a yellow segment, a gray segment, an orange segment, and a white segment. In yet further embodiments, the patch 302 includes one or more segments of patterns in black and white and/or color.

The patch 302 may also include a coded segment. For example, the patch 302 may include a numerical code, a bar code 306 (as shown in FIG. 3B), or a two-dimensional code 308, such as a QR code or an Aztec code (as shown in FIG. 3C and 3F). The code may include information about the patch 302 (e.g., model number), the patient, or the purchaser of the patches 302. According to an embodiment, the patch 302 may be customizable to the patient, and patient information (e.g., patient identifying information, patient medical information and insurance information) can be included in the code when the coded segment is produced (e.g., printed on the patch 302). For example, a bar code 306 as shown in FIG. 3B can be used to correctly identify a patient whose condition is being monitored and to store the image in the correct patient care file. If the patch 302 is used by a first response team, the code may include information about the emergency dispatch call or may inform the first responder with patient information such as patient history and drug allergies. According to exemplary embodiments, the code (e.g., QR code or Aztec code) may also include a Uniform Resource Locator address readable by a handheld device or a telehealth system, which may be used to connect to a remote healthcare provider (e.g., a telehealth service). A patch 302 may incorporate a radio frequency identification (RFID) component that includes some or all of the data discussed above. While an image of the patch will not display any information stored as part of the RFID component, a correlation between the visible data and an accessible database of RFID information may be used to cross-reference and obtain such data.

The wound management patch 302 can be of different shapes and sizes and can be manufactured using different materials. For example, a flexible patch similar to adhesive bandages may be more suitable to a single-use application, whereas a more rigid patch 302 that better retains its shape may be more suitable to a multi-use application. Alternatively, special shapes and sizes may be configured for particular parts of the body, such as smaller patches 302 for fingers or toes, or for use on children. The patch 302 may include any number of suitable materials, such as vinyl, polyethylene, polypropylene, polyester, polyurethane or fabric, among other things. In some embodiments, the patch 302 may also include an antimicrobial compound, such as an antibiotic coating, silver, copper, or a copper alloy.

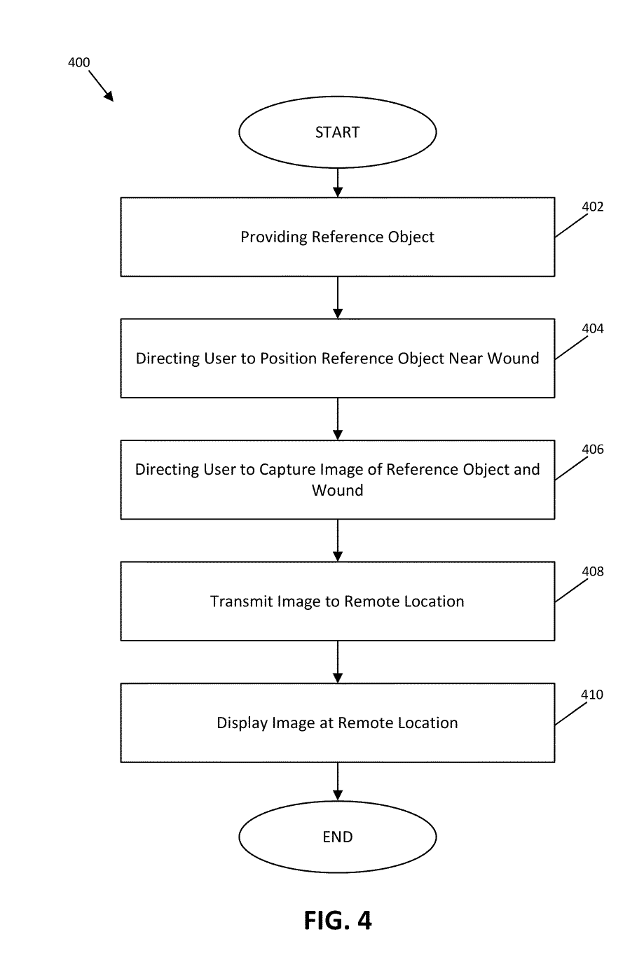

FIG. 4 illustrates an embodiment of a method 400 for wound management. The method 400 embodied in FIG. 4 begins with a providing reference object operation 402. In the providing reference object operation 402, a reference object such as that described herein is provided to the patient. The reference object may be mailed to the patient, provided with a patient monitoring device, provided by a caregiver, or purchased by the patient from a retailer.

Flow then proceeds to a placement operation 404 wherein the user is instructed to position the reference object near the wound. In the placement operation 404, the patient monitoring device may instruct the user to position the reference object in proximity to the wound, for example, two to six inches away from the edge of the wound. In other embodiments, the user is directed to place the reference object on a flat surface near the wound such that an optimal and even amount of light may reach the reference object. This may be done by displaying commands to the user such as “Please place the reference object near the wound”, or by asking questions confirming the user's actions such as “Please confirm that the reference object is placed as shown in the illustration.”

Flow then proceeds to directing the user to an image capture operation 406. In an embodiment, the image capture operation 406 may include instructing the user to capture an image of the reference object and the wound. For example, this may be done with a single image using an image capturing device. In some embodiments, the image capturing device is a digital camera, a video camera, or a cell phone camera. The image capture device may be incorporated into other devices such as a tablet PC, a PC, a monitoring device in a telehealth system, or the like. In some embodiments, the system may instruct the user to use a standardized light applicator before capturing the image in order to deliver light at specific wavelengths to produce accurate colors of the reference object and the wound.

The capture operation 406 may further include processing and post-processing of the captured image (or, alternatively, images or video depending on the embodiment) as described above. For example, in an embodiment the image capture operation 406 includes processing the image to confirm that the reference object is present in the image. This may include such things as confirming there is relatively little glare on the object by inspecting the uniformity of colors and readability (through character recognition) of text in different areas of the object. If colors deviate too much or if characters are not recognizable, in comparison to a predetermined threshold, the device may request the user to reposition the reference object, adjust the light or perform some other correction and take another image. The system may also confirm that the object is sufficiently facing the camera, such as by comparing the outlines of the object as shown in the image to information known about the actual object. For example, if a circular reference object appears on the image as a very stretched ellipse or a square reference object appears as a rectangle, an error may be shown to the user. A predetermined threshold of deviation from the actual shape may be used to determine when to display the error and request that the user make appropriate adjustments and repeat the process.

The capture operation 406 may include storing an image in memory along with additional metadata about the patient. The metadata may be generated by the capturing device and may incorporate information about the patient known to the device.

Flow then proceeds to a transmit image operation 408. In the transmit image operation 408, the user transmits the image to a remote location such as a remote healthcare provider workstation and/or a central processing unit for further processing. The image may be sent via text messaging, email, instant messaging, and/or uploading to a website that is accessible by a healthcare provider at a remote location. The image may be sent automatically or first displayed to the user to confirm that the image is acceptable and prompting the user to transmit the image if it is acceptable.

In the embodiment shown, a remote display operation 410 is also shown. In the remote display operation, the image received from the patient's device is displayed to a caregiver. Such display may include further processing of the image or automatic adjustment of the display settings of a monitor as described above. Alternatively, during the remote display operation 410 the viewing caregiver may be provided with an exact copy or replica of the reference object so that the caregiver may visually compare the actual object to the object as displayed in the image.

Those skilled in the art will recognize that the methods and systems of the present disclosure may be implemented in many manners and as such are not to be limited by the foregoing exemplary embodiments and examples. In other words, functional elements being performed by a single or multiple components, in various combinations of hardware and software or firmware, and individual functions, can be distributed among software applications at either the client or server level or both. In this regard, any number of the features of the different embodiments described herein may be combined into single or multiple embodiments, and alternate embodiments having fewer than or more than all of the features herein described are possible. Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software or firmware components described herein as would be understood by those skilled in the art now and hereafter.

Numerous other changes may be made which will readily suggest themselves to those skilled in the art and which are encompassed in the spirit of the disclosure and as defined in the appended claims. While various embodiments have been described for purposes of this disclosure, various changes and modifications may be made which are well within the scope of the present application. Numerous other changes may be made which will readily suggest themselves to those skilled in the art.

We claim:

1. A wound management kit comprising:
a pack of at least one reference object, the at least one reference object comprising a sheet of material of a known size including at least two colored segments, indicia on each reference object providing at least one of: a date identifier and a user identifier; and an adhesive to allow the at least one reference object to temporarily stick to an individual's skin.

2. The wound management kit of claim 1, wherein the pack includes ten or more reference objects.

3. A wound management system comprising:
a first reference object, the first reference object comprising a sheet of material of a known size including at least one colored segment; and
a camera for capturing an image of the first reference object in close proximity to a wound on the individual.

4. The wound management system of claim 3, wherein the first reference object further comprises an adhesive to allow the first reference object to temporarily stick to an individual's skin.

5. The wound management system of claim 3, further comprising a monitor for viewing the image.

6. The wound management system of claim 3, further comprising a processor for correcting the image based on the first reference object.

7. The wound management system of claim 5, wherein the monitor is remotely located.

8. The wound management system of claim 7, further comprising:
a second reference object available to a healthcare professional and near the remote monitor, allowing the healthcare professional to visually compare the second reference object with the first reference object while viewing the image.

9. The wound management system of claim 7, wherein the camera is part of a computing device adapted to transmit the image via a communication network to the monitor.

10. A wound management system comprising:
a first reference object, the first reference object comprising a sheet of material of a known size including at least one colored segment;
a camera for capturing an image of the first reference object in close proximity to a wound on the individual; and
a processor configured to recognize the first reference object and correct the image for at least one of color and size based on the first reference object.

11. The wound management system of claim 10, wherein the processor is configured to:
determine whether the first reference object is in the image; and
when the first reference object is not in the image, return an error.

12. The wound management system of claim 10, wherein the processor is remotely located.

13. The wound management system of claim 10, wherein the processor automatically corrects the image for both color and size.

14. The wound management system of claim 10, wherein the camera and the processor are part of a computing device.

15. The wound management system of claim 10, further comprising a monitor for viewing the corrected image.

16. The wound management system of claim 10, wherein the first reference object includes indicia providing at least one of: a date identifier and a user identifier.

17. The wound management system of claim 10, wherein the first reference object further comprises an adhesive to allow the first reference object to temporarily stick to an individual's skin.

18. The wound management system of claim 17, wherein the user identifier indicates at least one of: a name of the user and at least one medical condition associated with the user.

19. A method of wound management, the method comprising:
providing at least one reference object to a user;
directing the user to place the at least one reference object in close proximity to a wound;
directing the user to capture an image of the at least one reference object and the wound;
receiving the image at a monitor, and
displaying the image of the at least one reference object and the wound near a second reference object, the second reference object being identical in at least one of size, color and shape to the first reference object.

20. The method of wound management of claim 19, further comprising:
prompting the user to transmit the image over a network.

21. The method of wound management of claim 19, wherein the at least one reference object comprises an adhesive configured to adhere to skin.

22. The method of wound management of claim 19, wherein the at least one reference object comprises four colored segments.

23. The method of wound management of claim 19, wherein a first colored segment is blue, a second colored segment is red, and a third colored segment is green.

24. The method of wound management of claim 23, wherein the at least one reference object comprises at least one coded segment.

25. The method of wound management of claim 19, wherein the at least one coded segment comprises at least one of: numerical code, a bar code, and a two-dimensional code.

26. The method of wound management of claim 19, wherein the at least one reference object comprises at least one patterned segment.

27. A wound management kit comprising:
a pack of at least one reference object, the at least one reference object comprising a sheet of material of a known size including at least two colored segments, indicia on each reference object providing at least one of: a date identifier and a user identifier; and an adhesive to allow the at least one reference object to temporarily stick to an individual's skin.

28. The wound management kit of claim 27, wherein the pack includes ten or more reference objects.
